(19) United States
(12) Patent Application Publication
(10) Pub. No.: US 2024/0189556 A1
(43) Pub. Date: Jun. 13, 2024

(54) DEVICES, SYSTEMS, AND METHODS FOR REGULATING FLUID DELIVERY FOR ACCESSING A BODY LUMEN

(71) Applicant: BOSTON SCIENTIFIC SCIMED, INC., MAPLE GROVE, MN (US)

(72) Inventors: Scott Edward Corbeil, Litchfield, NH (US); Barry Weitzner, Acton, MA (US)

(73) Assignee: BOSTON SCIENTIFIC SCIMED, INC., Maple Grove, MN (US)

(21) Appl. No.: 18/530,544

(22) Filed: Dec. 6, 2023

Related U.S. Application Data
(60) Provisional application No. 63/430,748, filed on Dec. 7, 2022.

(51) Int. Cl. A61M 29/02; A61B 1/273; A61B 1/32; A61B 18/14; A61M 5/00; A61M 25/00

(52) U.S. Cl. CPC A61M 29/02; A61B 1/273; A61B 1/32; A61B 18/1485; A61M 5/007; A61M 25/0043; A61B 2018/00601; A61B 2018/144; A61M 2206/12; A61M 2210/1042; A61M 2210/1057

(57) ABSTRACT

A fluid delivery device and system configured to regulate delivery of fluid through a fluid delivery port to a target site within a patient, such as to identify, locate, dilate, etc., an opening in a tissue wall at the target site. A side port is defined in the wall of the fluid delivery device adjacent to but upstream of the fluid delivery port. If the fluid delivery port is partially or fully obstructed by contacting tissue, the side port provides a relief valve reducing the pressure of fluid from the fluid delivery port impacting the contacted tissue. The fluid flow path upstream of and adjacent to the side port may be tapered to create a Venturi effect which may cause entrainment of excess fluid at the target site into the fluid delivery device. Such entrained fluid may be recycled out the fluid delivery port.

### CROSS-REFERENCE TO RELATED APPLICATIONS

This application claims the benefit of priority under 35 U.S.C. § 119 to U.S. Provisional Application No. 63/430,748, filed Dec. 7, 2022, the entire disclosure of which is hereby incorporated by reference herein for all purposes.

### FIELD

The present disclosure relates generally to the field of medical devices, systems, and methods for accessing a body lumen. More particularly, the present disclosure relates to medical devices, systems, and methods for using fluid, such as compressible fluid, to access a body lumen. Even more particularly, the present disclosure relates to medical devices, systems, and methods for regulating fluid delivered to a target site to access a body lumen.

### BACKGROUND

Various medical procedures involve accessing a body passage or lumen within a patient. Certain endoscopic cannulation procedures may require advancing a guidewire and/or endoscopic accessory tool (e.g., sphincterotome, cannula, catheter, or the like) against, into, or through challenging patient anatomies. Although a guidewire may facilitate delivery of further medical instruments, not only by guiding the further medical instrument over the guidewire, but also facilitating access to a body passage or lumen, medical professionals sometimes face significant technical challenges when accessing certain anatomical structures even with a guidewire. In some instances, a target body lumen may be oriented at a difficult angle relative to the endoscopic accessory tool, may have a very small or sealed opening, or may include a tortuous anatomy, blockages (e.g., stones, or the like), or benign or malignant structures. To facilitate smooth and efficient entry of a guidewire and/or a medical instrument (e.g., an endoscopic accessory tool) into/through a target body lumen, medical professionals may manually rotate, oscillate, linearly advance, and/or reciprocate the endoscopic accessory tool, and, by proxy, the guidewire itself, to “wiggle” the guidewire and endoscopic accessory tool against, into, or through the body lumen with frictional forces exerted against the opening or tissue wall of the body lumen. Precise control of the force of movement imparted to the accessory tool and guidewire largely remains crude and uncontrolled. Even experienced medical professionals often require multiple attempts to achieve successful opening or entering of body lumens, especially when working against the natural friction and abnormal patient-specific pathologies of the specific body lumen. The likelihood of causing trauma to the tissues comprising or surrounding the target body passageway typically increases with the number of opening or entry attempts. In some instances, the medical professional may be required to abort the procedure entirely. In other instances, the traumatized tissues may be prone to harmful and potentially life-threatening post-operative inflammation. Improvements to devices, systems, and methods for accessing body lumens or passages within a patient thus would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a fluid delivery device has a primary fluid flow path for delivering fluid to a target site within a patient. The fluid exits the fluid delivery device via a fluid delivery port at the distal end of the primary fluid flow path which is optionally at or along the distal end of the fluid delivery device. The fluid may be pressurized to identify, locate, dilate, etc., an opening in a tissue wall at the target site. Further in accordance with various principles of the present disclosure, the fluid delivery device includes a secondary fluid flow path. The secondary fluid flow path may be in fluid communication with the primary fluid flow path. The secondary fluid flow path may be formed through a wall of the fluid delivery device, such as transverse to the primary fluid flow path. The primary fluid flow path generally extends longitudinally through the fluid delivery device. The secondary fluid flow path may form a pressure relief path for fluid flowing through the primary fluid flow path if the fluid delivery port is blocked or partially blocked (e.g., by tissue). In such instance, fluid is redirected to exit the fluid delivery device via the secondary fluid flow path (rather than from the fluid delivery port at the distal end of the primary fluid flow path). In some embodiments, the primary fluid flow path is tapered (the diameter decreases in a direction of downstream fluid flow) at a location upstream of the secondary fluid flow path. Such decrease in the diameter of the primary fluid flow path generates a Venturi effect increasing fluid velocity and reducing fluid pressure. The reduction in fluid pressure allows entrainment of fluid outside the fluid delivery device and in the area of the target site to be entrained into the fluid delivery device. Such entrainment regulates the volume of fluid delivered to the target site. Additionally or alternatively, such entrainment allows recycling of fluid delivered by the fluid delivery device, which may reduce the overall volume of fluid delivered to the target site.

In accordance with various principles of the present disclosure, a fluid delivery device is configured to deliver pressurized fluid through a fluid delivery port at an end thereof to a target site within a patient. The fluid delivery device defines therethrough a fluid delivery lumen fluidly couplable with a pressurized fluid source, and at least a first additional lumen configured for delivery of one of a device, a contrast fluid, a guidewire, or a wire filament configured for cutting tissue; and a side orifice through a wall of the fluid delivery device defining the fluid delivery lumen, the side orifice defined proximal to the fluid delivery port and adjacent thereto to provide pressure relief upon partial or complete obstruction of the fluid delivery port.

In some aspects, the fluid delivery lumen extends longitudinally along the fluid delivery device and the fluid delivery port extends out a distal end of the fluid delivery device to deliver fluid longitudinally out the fluid delivery device.

In some aspects, the fluid delivery lumen has a first diameter from a proximal end of the fluid delivery device to a narrowed region within the fluid delivery lumen having a second diameter smaller than the first diameter. In some embodiments, the narrowed region is upstream of the side orifice. In some aspects, the narrowed region creates a Venturi effect causing entrainment of fluid at the target site into the side orifice. In some aspects, the entrained fluid is redirected back out the fluid delivery port of the fluid delivery system.

In some embodiments, the first additional lumen is configured for passage of a guidewire therethrough and a further instrument over the guidewire to the target site.

In some embodiments, a second additional lumen is defined through the fluid delivery device configured for delivery of one of a device, a contrast fluid, a guidewire, or a wire filament configured for cutting tissue. In some embodiments, a third additional lumen is defined through the fluid delivery device configured for delivery of one of a device, a contrast fluid, a guidewire, or a wire filament configured for cutting tissue. In some aspects, the first additional lumen is configured for passage of a guidewire therethrough and a further instrument over the guidewire; the second additional lumen is configured for passage therethrough of a wire filament configure for cutting tissue; and the third additional lumen is configured for delivery therethrough of a contrast fluid to the target site.

In some embodiments, the side orifice is in fluid communication with a return lumen to provide pressure relief upon partial or complete obstruction of the fluid delivery port by delivering fluid outside the patient via the return lumen.

In accordance with various principles of the present disclosure, a system, for performing a procedure past an orifice within a patient's body, includes a fluid delivery device defining a fluid delivery lumen therethrough ending at a distal fluid delivery port, the fluid delivery device having a side port proximal to the fluid delivery port and adjacent thereto to provide pressure relief upon partial or complete obstruction of the fluid delivery port; and an additional instrument configured to be inserted along the fluid delivery device and inserted through the orifice within the patient's body.

Optionally, the system further includes a delivery device comprising a tubular delivery element configured to deliver the fluid delivery device therethrough.

In some embodiments, the fluid delivery lumen is couplable with a pressurized fluid source; and the diameter of the fluid delivery lumen decreases upstream and adjacent the side port to create a Venturi effect causing entrainment of fluid at the target site into the side port.

In some embodiments, the fluid delivery device defines an additional lumen therethrough for delivery of the additional instrument therethrough.

In accordance with various principles of the present disclosure, a method of regulating fluid delivered to a target site within a patient includes advancing a distal end of a fluid delivery device to the target site; directing a fluid delivery port toward tissue at the target site; delivering fluid through the fluid delivery port; and regulating the fluid delivered through the fluid delivery port via a side port defined in the fluid delivery device.

In some aspects, regulating the fluid delivered through the fluid delivery port includes diverting fluid through the side port upon the fluid deliver port contacting tissue at the target site to relieve fluid pressure on the contacted tissue.

In some embodiments, the diameter of the fluid delivery lumen is tapered upstream of the side port and regulating the fluid delivered through the fluid delivery port includes entraining fluid delivered by the fluid delivery port to the target site into the side port and recycling the entrained fluid out the fluid delivery port.

Optionally, the method further includes delivering fluid through the fluid delivery port to cause an opening in a tissue wall at the target site to dilate.

Optionally, the method further includes delivering an instrument to the dilated opening in the tissue wall and through the dilated opening.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, with redundant description omitted. For purposes of clarity and simplicity, not every element is labeled in every figure, nor is every element of each embodiment shown where illustration is not necessary to allow those of ordinary skill in the art to understand the disclosure. Moreover, a group of similar elements may be indicated by a number and letter, and reference may be made generally to one or such elements or such elements as a group by the number alone (without including the letters associated with each similar element).

The detailed description will be better understood in conjunction with the accompanying drawings, wherein like reference characters represent like elements, as follows:

FIG. 1 illustrates an elevational view of an example of an embodiment of a fluid delivery device and system formed in accordance with various principles of the present disclosure positioned in a schematic representation of a gastrointestinal environment.

FIG. 2 illustrates a detail view along detail area A of the example of an embodiment of fluid delivery device illustrated in FIG. 1, showing fluid pressure relief through the device.

FIG. 3 illustrates a detail view along detail area A of the example of an embodiment of fluid delivery device illustrated in FIG. 1, showing fluid entrainment into the device.

FIG. 4 illustrates a detail view, in cross-section, of a distal region of the example of an embodiment of a device illustrated in FIG. 2 or FIG. 3, along line IV-IV.

FIG. 5 illustrates an end view of the example of an embodiment of a fluid delivery device illustrate in FIG. 2 or FIG. 3.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” of a device is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site, and is not limited to just target tissue.

Devices, systems, and methods formed in accordance with various principles of the present disclosure assist with accessing an anatomical opening without the need or use of a medical instrument to touch, abrade, compress, or otherwise contact tissue surrounding and/or forming the opening. The anatomical opening may be an entrance to an anatomical structure such as a lumen, passageway/passage, duct, cavity, space, etc., to be accessed, reference being made interchangeably to such structures without intent to limit. It will be appreciated that reference may be made interchangeably herein, and without intent to limit, to anatomical openings, entrance, orifices, etc., leading to other anatomical structures to be accessed. Devices, systems, and methods formed in accordance with various principles of the present disclosure facilitate identification and/or dilation of the anatomical opening (and optionally also the anatomical structure into which the anatomical opening leads) to facilitate access thereto. It will be appreciated that reference to a body opening and/or passage includes naturally-existing openings/passages (e.g., the colon) as well as medically-created openings/passages (e.g., a passage created with the use of a medical instrument, and not existing without medical intervention) or otherwise. Furthermore, it will be appreciated that devices, systems, and method disclosed herein may be used within a lumen or duct, regardless of the location of an anatomical opening with respect thereto.

More particularly, various principles of the present disclosure are applicable to devices, systems, and methods which use a fluid (e.g., a gas such as air, nitrogen, carbon dioxide, etc.), such as a compressible fluid, delivered via a fluid delivery device to identify and/or access and/or dilate an anatomical opening at a target site. The fluid delivery device may include an elongate flexible tubular element or other suitable medical device navigable within a body (e.g., transluminally) to an internal anatomical opening (e.g., the papilla of Vater) within a patient. It will be appreciated that reference may be made herein interchangeably to identifying, accessing, dilating, etc., an anatomical opening without intent to limit. The fluid may be delivered from a pressurized gas source to the fluid delivery device and out a fluid delivery port of the fluid delivery device. A jet of the fluid is directed towards or onto tissue by the fluid delivery device (e.g., through the delivery port thereof) with sufficient force to at least partially open or dilate the anatomical opening with the fluid. A guidewire, catheter (e.g., delivery catheter), endoscope (e.g., duodenoscope, cholangioscope, etc.), endoscopic accessory device, or other further medical instrument, device, tool, etc., may then be advanced to perform a procedure with respect to the target site, such as with respect to an anatomical structure past the anatomical orifice opened by the fluid delivered by the fluid delivery device. It will be appreciated that terms such as tools, instruments, devices, etc., may be used interchangeably herein without intent to limit. The fluid delivery device may be a fluid-delivery catheter or other tubular element including one or more lumens through which fluid as well as one or more additional devices may be delivered to the target site. The fluid delivery device may be an elongate flexible tubular element. In some instances, the medical device used to perform the procedure and/or a different medical device is advanced to make physical contact with the target anatomical structure or tissue surrounding the opening such as to access anatomical structure beyond the opening. In some instances, it may be desirable to maintain the anatomical opening in an opened or dilated configuration, yet limit an amount of fluid delivered to the target site.

Generally, it is desirable to exhibit care in directing a fluid jet with respect to an anatomical target site within a patient. For instance, it is generally desirable to exhibit care in directing a fluid delivery port of the fluid delivery device through which the fluid jet is delivered to regulate the fluid pressure impacting the tissue at the target site. There is an inherent risk that if or when the fluid delivery port (e.g., at the distal end of the fluid delivery device) makes direct contact with tissue (particularly complete circumferential contact), the imparted pressure and volumetric flow from the exiting fluid jet could result in local tissue injury, or gas infiltration into the tissue. Furthermore, if the fluid delivery port is positioned partially or fully within the anatomical structure to be accessed (beyond the anatomical opening), the pressure and/or active fluid flow through the fluid delivery device may result in tissue injury, barotrauma, rupture or other undesirable effects along the body lumen, duct, cavity, etc., and/or to tissues and/or organs downstream of the device.

Moreover, often times the target site and/or anatomical structure to be accessed exists within enclosed or normally enclosed anatomical spaces or cavities. Although there is an advantage to using a jet of fluid to identify and/or to dilate the entrance to a body lumen or duct, generally there is also a desire/need to limit the overall or total amount of fluid delivered to the enclosed, or normally enclosed space in which the clinician is operating. Delivery of relatively large volumes of fluid to an enclosed or normally-enclosed anatomical structure or region (e.g., cavity, lumen, etc.) may require extended periods of time for the fluid to be absorbed by the patient's body. While the excess fluid resides within the patient, the patient may feel pain, discomfort, or abnormal sensations, generally until the gas has been fully absorbed or otherwise passed. In extreme cases, the excess fluid may cause barotrauma, disruption, rupture, or other undesirable effects.

In accordance with various principles of the present disclosure, the fluid delivery device is configured to minimize risk of excess fluid pressure impinging on anatomical tissue and/or entering the anatomy. In particular, the flow path through the fluid delivery device may be configured to minimize risk of excess fluid pressure impinging on anatomical tissue and/or entering the anatomy. For instance, in some aspects, the fluid delivery device has a primary fluid flow path as well as an additional fluid flow path configured to reduce excess fluid pressure and/or fluid, in general, delivered by the primary fluid flow path fluid to and out the fluid delivery port to the target site. The additional fluid flow path through the fluid delivery device may redirect pressurized fluid to not impact tissue contacted by the fluid delivery device and/or to entrain excess fluid at the target site into the catheter.

More particularly, in accordance with various principles of the present disclosure, the fluid flow path through a fluid delivery device configured to deliver fluid to a target site is designed with a unique geometry for regulating fluid flow with respect to the target site. The fluid flow path through the fluid delivery device may regulate the delivery of fluid to and/or through a fluid delivery port of the fluid delivery device and to the target site, and/or regulate fluid delivery within and/or with respect to the target site. For example, the fluid flow path through the fluid delivery device may be provided with a unique geometry within and along a region thereof. In some embodiments, the unique geometry is provided along a distal region of the fluid delivery device. In some embodiments, the unique geometry is provided in the vicinity of the fluid delivery port. The fluid delivery port may be along a distal region of the fluid delivery device, such as at a distal end thereof. The unique geometry may be provided along a distal region of the fluid delivery device in the vicinity of the fluid delivery port. In some embodiments, the unique geometry includes a converging, or converging-diverging geometry along the fluid flow path through the fluid delivery device.

In some embodiments, the fluid delivery device is configured to have a primary fluid flow path and a secondary fluid flow path. The secondary fluid flow path establishes fluid communication between the primary fluid flow path (typically extending longitudinally through the fluid delivery device to the fluid delivery port) and the external environment of the fluid delivery device. The secondary fluid flow path may be through an orifice provided through the wall of the fluid delivery device. The orifice may be alternately referenced herein as a port or side port, hole, opening, aperture, duct, passage, etc., such terms being used interchangeably herein without intent to limit. It will be appreciated that the secondary fluid flow path may, at least initially, be transverse to the primary fluid flow path, and may simply be the path through the orifice in the wall of the fluid delivery device. The secondary fluid flow path may provide an outlet for fluid flow if the fluid delivery port of the fluid delivery device is obstructed or blocked, such as by tissue at the target site. Such outlet may be helpful in preventing or at least reducing the impact of high pressure fluid against tissue at the target site, such as tissue contacted by the fluid delivery device and obstructing or blocking the fluid delivery port thereof. By relieving pressure and diverting high-pressure fluid flow, the risk of local tissue injury and gas infiltration, as well as the risk of downstream tissue/organ injury, damage, and/or rupture, etc., is significantly reduced. Additionally, the potential for device rupture or failure from elevated back pressures, which could result in damage to other devices in use, and/or injury to the patient or the user, are also avoided.

In some embodiments, the secondary fluid flow path is provided along a narrowed-diameter region of the primary fluid flow path through the fluid delivery device. More particularly, in some embodiments, the primary fluid flow path has a first diameter from a proximal end to a tapered region, at which the primary fluid flow path tapers to a second diameter smaller than the first diameter. The reduction of the diameter of the primary fluid flow path causes an increase in the velocity of the fluid flow therethrough, creating a Venturi effect with an accompanying reduction in fluid pressure in such region. Such tapering of the primary fluid flow path and resultant Venturi effect is upstream to the secondary fluid flow path. As fluid is directed distally through the primary fluid flow path, past the secondary fluid flow path, and out the fluid delivery port, excess fluid delivered by the fluid delivery device is entrained into the fluid delivery device via the secondary fluid flow path. As such, the fluid delivery device formed in accordance with various principles of the present disclosure directs a jet of fluid to the target site (such as onto target tissue to dilate/open an anatomical opening at the target site), and a portion of the fluid being delivered to the target site is entrained, via the Venturi mechanism, from the enclosed or normally enclosed space in which the clinician is operating and into the fluid delivery device. A portion of the fluid delivered by the fluid delivery device is thus recycled back into the fluid delivery device, thereby controlling the total volume of fluid delivered to the target site within the patient and/or ultimately reducing the total volume of new/additional fluid being added to the enclosed or normally enclosed space at the target site. The secondary fluid flow path may also serve as a pressure relief mechanism, as described above, if the fluid delivery port of the fluid delivery device is obstructed or blocked. In such instance, the secondary fluid flow path allows pressure relief and flow diversion in a direction opposite the direction of the Venturi effect. As may appreciated, a combination Venturi and pressure/flow relief mechanism is thereby provided in such embodiment.

A fluid delivery device formed in accordance with various principles of the present disclosure may be advantageously used as a cannulation catheter for accessing a desired target through an anatomical opening which is difficult to locate and/or identify and/or access. The fluid delivery device may be used with additional medical devices and/or with medical systems (e.g., endoscopic devices, accessory tools, and/or guidewires inserted through an endoscope, duodenoscope, cholangioscope, etc.) to gain selective access to, alignment with, and/or cannulation of a narrow anatomical structure (e.g., narrow body passageway). For example, devices, systems, and methods formed in accordance with various principles of the present disclosure may be useful for diagnostic or therapeutic procedures in the biliary and/or pancreatic tracts, among being useful for other purposes. Access to the pancreaticobiliary system, as facilitated by devices, systems, and methods disclosed herein, may be required to diagnose and/or treat a variety of conditions, including but not limited to tumors, gallstones, infection, sclerosis, and pseudocysts. The devices, systems, and methods disclosed herein may also be useful for navigation in other parts of the body such as the cardiovascular system and so forth.

One example of a use of a fluid delivery device formed in accordance with various principles of the present disclosure is for cannulating the biliary tree, such as through the major duodenal papilla. An endoscopic retrograde cholangiopancreatography (ERCP) procedure or a cholangioscopy procedure may then be performed to examine the bile ducts, pancreas, liver, and/or gallbladder. Endoscopic retrograde cholangiopancreatography (ERCP) may be used to diagnose and treat conditions of the common bile duct, including, for example, gallstones, inflammatory strictures, leaks (e.g., from trauma, surgery, etc.), cancer, and/or other conditions. In an ERCP procedure, a physician may view, through an endoscope, the inside of the stomach and/or the duodenum. During the procedure, dyes may be injected into the ducts in the biliary tree and/or pancreas so that the area can be seen using X-rays. These procedures may necessitate gaining and maintaining access to the papilla of Vater, the common bile duct, and/or the pancreatic duct, which may be technically challenging, may require extensive training and practice to gain proficiency, and may require one or more expensive and/or complex tools in order to perform.

During an ERCP procedure, a number of steps may be performed while the subject is sedated and/or anaesthetized. For example, an endoscope may be inserted through the mouth, down the esophagus, into the stomach, through the pylorus into the duodenum, and to a position at or near the papilla of Vater (also referred to as the ampulla of Vater), which is the opening of the common bile duct and the pancreatic duct into the duodenum (the access entrance into the common bile duct and pancreatic duct from the duodenum/outlet of such ducts into the duodenum). Due to the shape of the papilla, and the angle at which the common bile duct and pancreatic duct meet the wall of the duodenum, the distal end of the endoscope is generally placed just past the papilla. Due to the positioning of the endoscopes beyond the papilla, the endoscopes typically used in these procedures are side-viewing endoscopes, such as duodenoscopes. The side-viewing feature provides imaging along the lateral aspect of the distal end rather than from an axial aspect at a terminal end of the endoscope. Such orientation may allow a clinician to obtain an image of the medial wall of the duodenum, where the papilla of Vater is located, even though the distal tip of the endoscope is beyond the opening.

Devices and systems formed in accordance with various principles of the present disclosure may be delivered through a working channel of an endoscope to the target site. When a fluid delivery device formed in accordance with various principles of the present disclosure is delivered through a duodenoscope, an elevator mechanism of the duodenoscope may be used to manipulate (raise, lower, etc.) a distal end of the fluid delivery device to direct the fluid delivery port thereof to tissue at the target site to identify, locate, access, dilate, etc., the anatomical passage through which access is desired, such as the papilla of Vater. Fluid can be delivered by the fluid delivery device for delivery to a target site within the patient in any suitable manner. Some example techniques and systems for delivering fluid to an opening of a body lumen are described in U.S. Patent Application Publication No. 2019/0380565, which was filed on Jun. 13, 2019, and titled Devices, Systems And Method For Accessing A Body Lumen, which application is hereby incorporated herein by reference in its entirety for any and all purposes. Once the anatomical opening has been located, identified, dilated, accessed, etc., an additional instrument may be inserted along the fluid delivery device, such as through the fluid delivery lumen therethrough.

It will be appreciated that although configurations of the present disclosure may be described with specific reference to the common bile duct (CBD) or pancreatic duct (PD), such as during an Endoscopic Retrograde Cholangiopancreatography (ERCP) procedure, it should be appreciated that such medical devices and systems may be used in a variety of other medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. Moreover, it should be appreciated that the disclosed medical devices and systems may be inserted via different access points and approaches, e.g., percutaneously, endoscopically, laparoscopically, or some combination thereof.

Various embodiments of devices, systems, and methods for using a fluid stream to identify, access, dilate, open, etc., anatomical structures at a target site, and for regulating fluid delivered to the target site, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

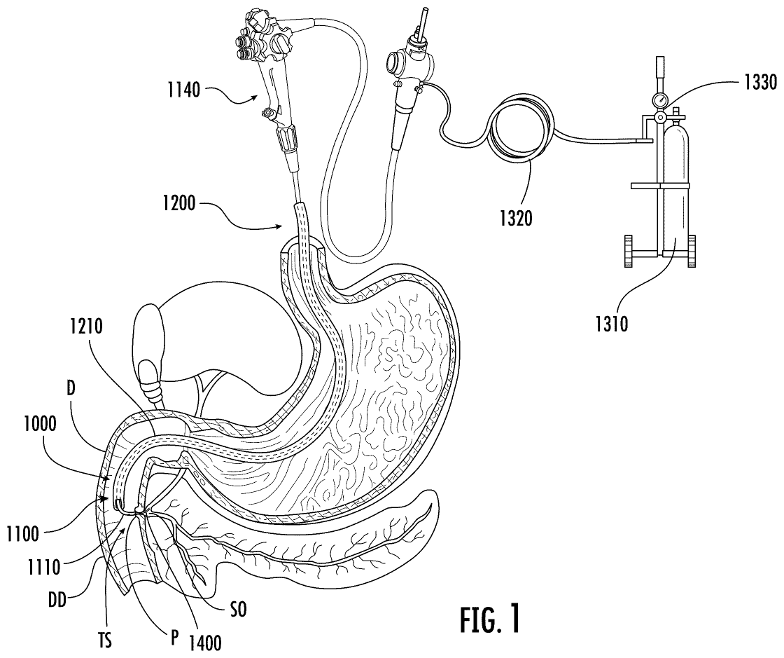

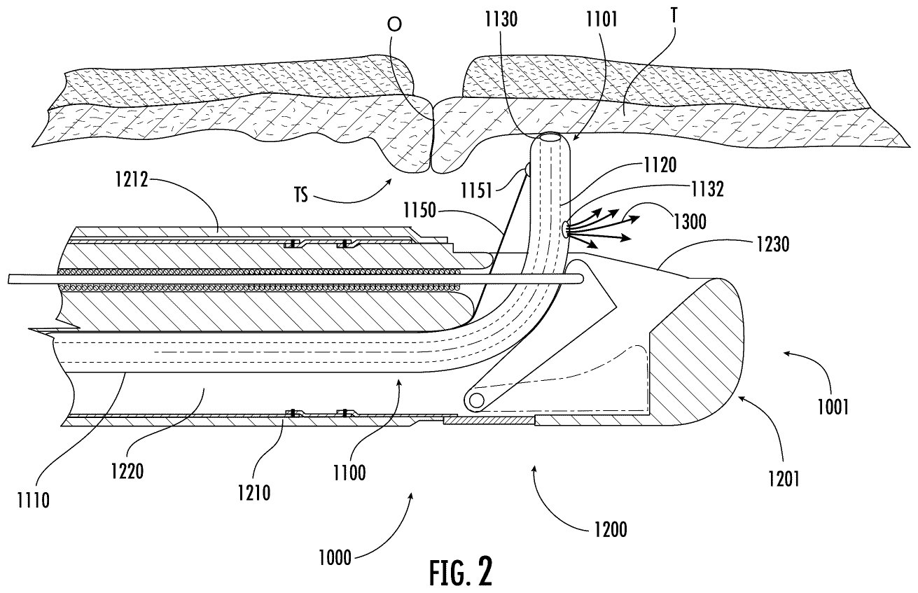

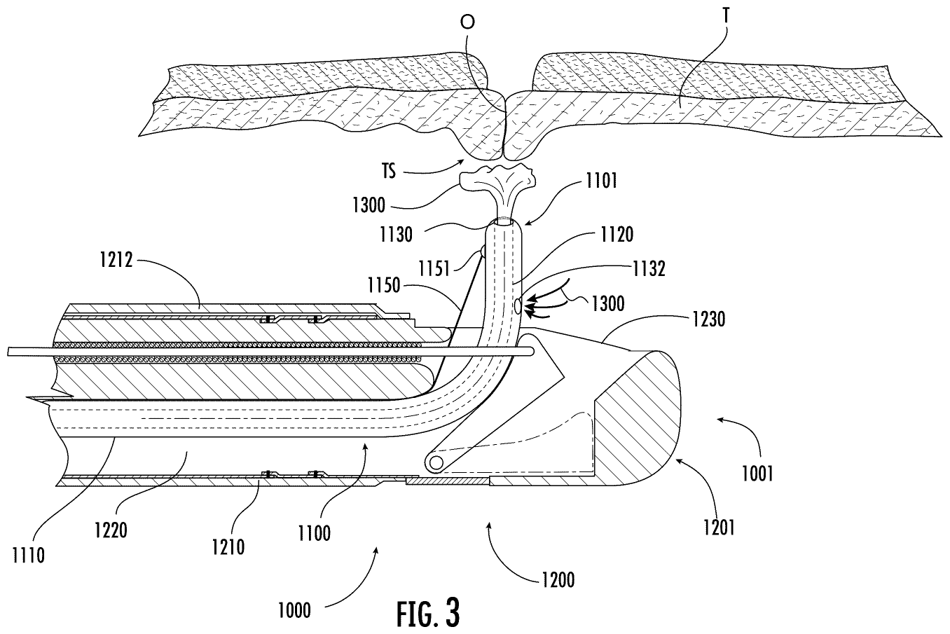

Turning now to the drawings, an example of an embodiment of a fluid delivery device 1100 and associated delivery system 1000 formed in accordance with various principles of the present disclosure is illustrated in FIG. 1. The delivery system 1000 optionally includes a delivery device 1200 configured to facilitate delivery of the fluid delivery device 1100 to a target site TS within a patient's body The illustrated example of an environment in which the illustrated examples of embodiments of a fluid delivery device 1100 and a delivery system 1000 are delivered and used is a gastrointestinal environment, although the fluid delivery device 1100 and delivery system 1000 (optionally with appropriate modifications thereto) may be delivered and used at or within other anatomical environments. Further details of the fluid delivery device 1100 and portions of the delivery system 1000 along detail area A of FIG. 1 are illustrated in FIG. 2 and FIG. 3.

As illustrated in FIG. 2 and in FIG. 3, the respective distal ends 1001, 1101 delivery system 1000 and the fluid delivery device 1100 are delivered to a target site TS at which an anatomical opening O is located. If a delivery device 1200 is used, the distal end 1201 typically is also delivered to the target site TS. The fluid delivery device 1100 has a tubular element 1110 defining a fluid delivery lumen 1120 defined therethrough to deliver fluid 1300 to the target site TS. More particularly, the illustrated fluid delivery lumen 1120 extends generally longitudinally through the tubular element 1110 of the fluid delivery device 1100, and is in fluid communication with and ends at a fluid delivery port 1130. The fluid delivery lumen 1120 delivers fluid 1300 along a flow path through the fluid delivery lumen 1120 and out of the fluid delivery device 1100 via the fluid delivery port 1130 to the target site TS. Fluid 13002 may be delivered to the fluid delivery device 1100 from a fluid source 1310 fluidly coupled with the delivery system 1000, as shown in FIG. 1 and as described in further detail below with reference to FIG. 5. The fluid 1300 is delivered from and flows from the fluid source 1310 through the fluid delivery lumen 1120 and out the fluid delivery port 1130 at a pressure sufficient to cause the fluid 1300 to impinge on tissue at the target site TS to cause the anatomical opening O to open (if partially or fully closed) or to otherwise facilitate identification of the location of the anatomical opening O. In the illustrated embodiment, the fluid delivery port 1130 is defined at the distal end 1101 of the fluid delivery device 1100. However, other locations of the fluid delivery port 1130 with respect to the fluid delivery device 1100 are within the scope and spirit of the present disclosure. The fluid delivery device 1100, or at least a distal portion thereof, may be manipulable in order to direct the fluid delivery port 1130 with respect to the target site TS to deliver fluid 1300 thereto in a manner in which the impingement of fluid 1300 on the tissue T at the target site TS facilitates identification of the location of the anatomical orifice O, such as in a manner described in further detail below.

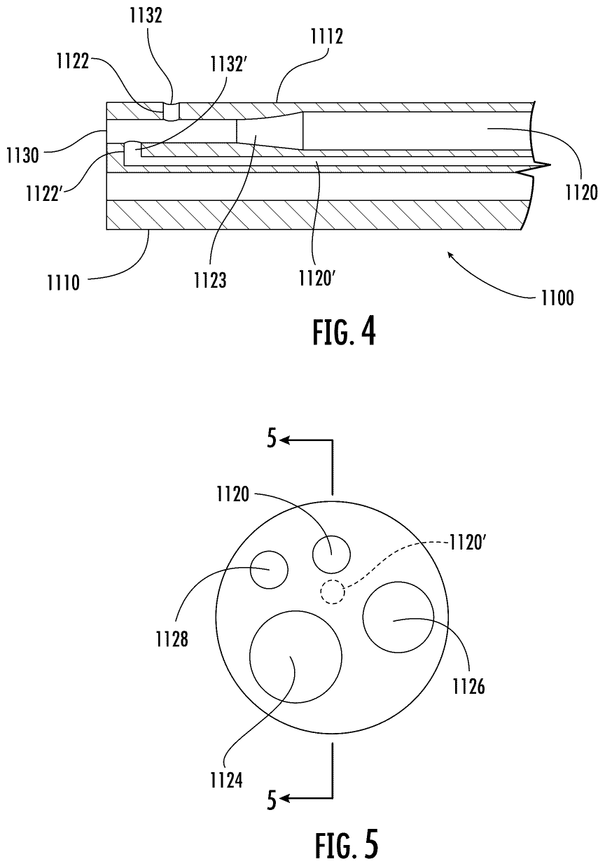

In accordance with various principles of the present disclosure, the fluid delivery device 1100 includes an additional orifice or port 1132 configured to enhance the functioning of the fluid delivery device 1100 in one or more manners. In the example of an embodiment of a fluid delivery device 1100 illustrated in FIG. 2 and FIG. 3, the additional port 1132 is a side port formed through the wall 1112 of the tubular element 1110 of the fluid delivery device 1100 (as may be appreciated with reference to the cross-sectional detail view in FIG. 4 of detail IV-IV in FIG. 2 and FIG. 3). However, other configurations are within the scope and spirit of the present disclosure. Moreover, in the example of an embodiment of a fluid delivery device 1100 illustrated in FIG. 2 and FIG. 3, the additional port 1132 is located in a distal region of the fluid delivery device 1100 sufficiently close to the distal end 1101 of the fluid delivery device 1100 to affect the fluid flow at the target site TS, as may be appreciated by one of ordinary skill in the art with reference to the various characteristics of the anatomical site, the fluid delivery device 1100, the fluid 1300, etc.

In the example of an embodiment illustrated in FIG. 2, the fluid delivery port 1130 of the fluid delivery device 1100 is provided at the distal end 1101 of the fluid delivery device 1100 at the end of the fluid delivery lumen 1120. An orifice in the wall 1112 of the tubular element 1110 of the fluid delivery device 1100 defines an additional lumen 1122 fluidly communicating with the additional fluid port 1132 proximal to the fluid delivery port 1130. The additional fluid port 1132 is sufficiently close to the fluid delivery port 1130 so that if the fluid delivery port 1130 contacts or partially contacts tissue T at the target site TS, the flow of fluid 1300 through the fluid delivery lumen 1120 is diverted through an additional flow path through the additional lumen 1122 and out the additional fluid port 1132 instead of flowing out through the fluid delivery port 1120 and impinging directly or substantially directly onto tissue T at the target site TS. The preferred or optimal location of the additional fluid port 1132 with respect to the fluid delivery port 1130 may be determined based on the specific geometry of the fluid delivery device 1100 and the fluid delivery lumen 1120 therethrough as well as the dimensions of the space at the target site TS at which the distal end 1101 of the fluid delivery device 1100 is to be navigated to direct fluid 1300 at tissue T. Additionally or alternatively, the diameter of the additional fluid port 1132 may be selected/tailored to completely divert fluid flow, or to partially divert fluid flow. With a known fluid delivery flow rate, the diameter of the additional fluid port 1132 may be selected to allow a selected level of pressure while relieving pressure above such level. As may be appreciated, the additional fluid port 1132 provides a pressure relief mechanism protecting tissue T from effects of impingement of high pressure fluid flow thereon.

As illustrated in FIG. 2, fluid diverted from the fluid delivery port 1130 to the additional port 1132 may be directed laterally out the wall 1112 of the tubular element 1110 of the fluid delivery device 1100 at a location spaced apart from the tissue T at the target site TS. As such, fluid pressure against tissue contacted by the fluid delivery port 1130 is reduced, averting potential deleterious consequences of high pressure fluid impinging on the tissue. Optionally, the fluid 1300 may exit through an additional port 1132′ and duct 1122′ directed through a return lumen 1120′ which vents the redirected fluid 1300 to outside the patient, such as to prevent overfilling of the target site TS with fluid 1300. An example of an embodiment of a return lumen 1120′, and associated port 1132′ and duct 1122′, is illustrated in the cross-sectional detail views in FIG. 4 and FIG. 5.

In accordance with various principles of the present disclosure, the additional port 1132 of the fluid delivery device 1100 may affect the volume of fluid 1300 within/delivered to the target site TS. For instance, the additional port 1132 may be configured to affect the volume of fluid 1300 within the target site TS by being configured to recycle fluid 1300 delivered by the fluid delivery lumen 1120. More particularly, in the example of an embodiment illustrated in FIG. 3, and in further detail in FIG. 4, the cross-sectional area of the fluid delivery lumen 1120 is reduced in a distal region thereof along a tapered section 1123 upstream of the additional lumen 1122 and additional port 1132. Such reduction in cross-sectional area causes an increase in fluid flow velocity and concurrent decrease in fluid pressure, creating a Venturi effect affecting pressure dynamics at the additional lumen 1122 and the additional fluid port 1132. Fluid 1300 within the target site TS, external to the fluid delivery device 1100 and in the region of the additional fluid port 1132 is thus entrained into the fluid delivery lumen 1120 via the additional fluid port 1132. The entrained fluid may be directed back out the fluid delivery port 1130 to the target site TS. As such, the volume of fluid 1300 delivered to the target site TS may be regulated and/or the total volume of fluid 11300 delivered to the target site TS may be regulated and/or reduced, with accompanying benefits to the patient. It will be appreciated that the reduction ratio of such tapered section 1123 will be proportional to the pressure reduction in this section and subsequently proportional to the volumetric fluid entrainment potential of this fluidic system.

The tapered section 1123 may be considered to differentiate the fluid delivery lumen 1120 into two or more sections. An upstream section from the proximal end of the fluid delivery lumen 1120 to the proximal/upstream end of the tapered section 1123 has a first diameter, the diameter of the fluid delivery lumen 1120 decreasing distally along the tapered section 1123. The diameter of the section of the fluid delivery lumen 1120 downstream of the tapered section 1123 (which may be a constant diameter, in contrast with the diminishing diameter of the tapered section 1123) may have a reduced diameter relative to the upstream section of the fluid delivery lumen 1120, such reduced diameter being the same as or smaller than the diameter of the tapered section 1123 at the distal end of the tapered section 1123 (typically the smallest diameter of the tapered section 1123) However, the diameter of the downstream section of the fluid delivery lumen 1120, downstream of the tapered section 1123, from the distal end of the tapered section 1123 to the fluid delivery port 1130, may increase in diameter to have a diameter equal to or even larger than the upstream section of the fluid delivery lumen 1120 diameter.

It will be appreciated that the above-described additional lumen 1122 creates a secondary flow path which provides fluid communication between the primary flow path through fluid delivery device 1100 and the external environment (e.g., target site TS) of the fluid delivery device 1100. The additional lumen 1132 provides an additional flow path through which fluid 1300 can move from the primary fluid flow path through the fluid delivery lumen 1120, through the wall 1112 of the tubular element 1110 of the fluid delivery device 1100, and to the environment outside the fluid delivery device 1100. Conversely, the additional lumen 1132 also provides an additional flow path from the space outside the fluid delivery device 1100 into the primary fluid flow path within the fluid delivery device 1100.

It will be appreciated that more than one side port may be provided in the wall 1112 of the tubular element 1110 of the fluid delivery device 1100 and in fluid communication with the fluid delivery lumen 1120. For instance, an additional lumen 1122′ and port 1132′ may be in fluid communication with a return duct and/or lumen (such as the return duct 1122′ and return lumen 1120′ illustrated in phantom in FIG. 4 and FIG. 5). Other additional ports may fluidly communicate the fluid delivery lumen 1120 with the environment outside the fluid delivery device 1100 (e.g., the target site TS) such as to entrain fluid into the fluid delivery lumen. Various other configurations and fluid communications of additional ports and lumens and combinations thereof are within the scope and spirit of the present disclosure, as may be appreciated by those of ordinary skill in the art. The one or more additional lumens through the wall 1112 of the tubular element of the fluid delivery device 1100 may be perpendicular to or at an acute angle to the longitudinal axis LA of the fluid delivery device 1100 and/or the fluid delivery lumen 1120 upstream of the additional port 1132.

It will be appreciated that principles of the present disclosure may be applied to various medical devices and systems delivering a fluid, such as a pressurized fluid, to a target site within a patient. The fluid may be delivered for a variety of purposes, such as to locate, identify, dilate, open, etc., an anatomical orifice. For instance, some medical procedures involve accessing a desired target along the biliary tree by advancing an endoscope through the duodenum to a position adjacent the papilla of Vater, and advancing a medical device, which may be a guidewire, through the endoscope and through the papilla of Vater to the intended target (e.g., the pancreatic duct or the common bile duct). In some instance, the opening to the lumen is difficult to locate, and, in some instance, may even be blocked. A stream of fluid directed at tissue in the vicinity of the opening may cause the opening to open in a less traumatic manner than by advancing a medical instrument along the tissue wall to locate the opening.

An example of an application of principles of the present disclosure is illustrated in FIG. 1, showing a delivery system 1000 with an example of an embodiment of a fluid delivery device 1100 (such as described above) having a tubular element 1110 through which fluid may be delivered to a target site TS within a patient's body (in this example, within a gastrointestinal system, such as adjacent the Sphincter of Oddi SO within the duodenum D). At least the tubular element 1110 of the fluid delivery device 1100 may be delivered with a delivery device 1200 (e.g., an endoscope, such as a duodenoscope). The fluid delivery device 1100 may be delivered through the tubular element 1210 (which may also be referenced as an insertion tube) of the delivery device 1200, such as through a working channel 1220 defined through the tubular element 1210, as illustrated in FIG. 2 and FIG. 3.

In the illustrated example of an embodiment, the delivery system 1000 is used to perform selective cannulation during an ERCP procedure. As such, the delivery device 1200 may be a duodenoscope with a tubular element 1210 having a lateral- or side-opening working channel 1220 defined therethrough, opening to a side port 1230 defined through the wall 1212 of the tubular element 1210, as illustrated in FIG. 2 and FIG. 3. The delivery device 1200 and the fluid delivery device 1100 are illustrated in FIG. 1 as being delivered through the duodenum D to a body lumen such as the major papilla P (e.g., ampullary entry) near the descending duodenum DD to access the Sphincter of Oddi Complex SO. The delivery device 1200 may include a control handle 1240, and the fluid delivery device 1100 may include a control handle 1140 each configured to facilitate maneuvering of the respective tubular element 1110, 1210 to the target site TS within the patient's body. The control handles 1140, 1240 may be provided with various ports and/or actuators/controllers (e.g., valves, buttons, dials, locks, knobs, etc.) such as known to those of ordinary skill in the art (and thus not individually labeled or discussed in detail herein) for delivery of other materials, devices, etc., to the target site TS. A guidewire 1400 may be advanced through the delivery system 1000 towards the major papilla P to facilitate guidance of an additional medical instrument towards and through the papilla P, such as to perform a procedure beyond the opening O into the papilla P. The guidewire 1400 may be advanced through a lumen of the fluid delivery device 1100 and then the fluid delivery device 1100 may be removed, leaving the guidewire 1400 in place as a rail to delivery other medical devices such as a stent delivery catheter, basket devices, etc.

As discussed above, accessing the papilla P may be difficult because the opening is small compared to many medical devices, the opening may be completely collapsed/closed, and/or the opening may extend into the descending duodenum DD at an angle that may be difficult to visualize and/or access. Thus, a medical professional may be required to manipulate the fluid delivery device 1100 and/or the guidewire 1400 by manually rotating the tubular element 1110 and/or the delivery device 1200 (e.g., with respective handles 1140, 1240) and/or using an elevator 1250 within the distal end of the tubular element 1210 of the delivery device 1200 adjacent the side port 1230 (such as illustrated in FIG. 2 and FIG. 3) in an attempt to better align or orient the fluid delivery device 1100 and/or guidewire 1400 with respect to the delivery device 1200 and the opening O of the papilla P. Difficult cannulation procedures in which the medical professional fails to access the Sphincter Papillae within a certain time limit, or after a certain number of unsuccessful attempts, as mentioned above, may lead to significant post-procedure complications, such as post-ECRP pancreatitis (PEP). Directing fluid via the fluid delivery device 1100 towards tissue T at the target site TS, in accordance with various principles of the present disclosure, reduces potential trauma to the tissue which may occur with conventional techniques of manipulating a medical device against or into the opening of the body lumen. As may be appreciated with reference to FIG. 1, fluid 1300 may be supplied from a fluid source 1310 (e.g., a CO₂ tank or a container of other suitable fluid) to the fluid delivery lumen 1120 of the fluid delivery device 1100 via tubing 1320. A pressure controller 1330 (e.g., a pressure adjustment knob and/or other suitable flow/pressure controller) may be operatively coupled with the fluid source 1310 and/or tubing 1320 and configured to control a pressure or flow rate (e.g., volumetric flow rate) of fluid 1300 to the target site TS. Although not required, a filter (e.g., a 0.1 micro filter and/or other suitable filter) may be provided in the flow path of the fluid 1300 (e.g., in fluid communication with the tubing 1320 and/or the fluid delivery lumen 1120 of the fluid delivery device 1100), such as distal to or downstream of the fluid source 1310 and proximal to or upstream of the fluid delivery lumen 1120.

In some embodiments, as illustrated in FIG. 2 and FIG. 3, a wire filament 1150 may extend along the fluid delivery device 1100, such as through a wire-filament-receiving lumen 1126 of the fluid delivery device 1100 (sec, e.g., FIG. 5) and may be energized for the purpose of cutting tissue within a subject. The wire filament 1150 may have a proximal end (not shown) adjacent and optionally operatively coupled with the handle 1140 of the fluid delivery device 1110 for actuation thereof, The distal end 1151 of the wire filament 1150 may be connected to the distal end 1101 of the fluid delivery device 1100. In some cases, an actuator of the handle 1140 of the fluid delivery device 1100 may be manipulated to slide the wire filament 1150 along the fluid delivery device 1100 (e.g., within the wire-filament-receiving lumen 1126 of the fluid delivery device 1100) to manipulate the wire filament 1150 as desired or needed. For instance, sliding the wire filament 1150 may result in the distal end 1101 of the fluid delivery device 1100 moving responsively to the sliding of the wire filament 1150. As such, the handle 1140 of the fluid delivery device 1100 may be manipulated to control or adjust a position of the distal end 1101 of the fluid delivery device 1100, such as with respect to the anatomical opening O at the target site TS. In some cases, the wire filament 1150 may be utilized to electrically cut, remove, and/or cauterize tissue along or around the anatomical opening O. As such, the handle 1140 or other suitable portion of the fluid delivery system 1000 may include an electrical connection for an energy source (e.g., a radiofrequency energy source, or the like) to energize the wire filament 1150 and facilitate cutting tissue. When the handle 1140 is manipulated to apply tension to the wire filament 1150 and adjust a position of the distal end 1101 of the fluid delivery device 1100, the wire filament 1150 may be spaced from the fluid delivery device 1100 and electrified to facilitate cutting tissue T at or adjacent to the opening O at the target site TS. Such cutting may be performed once the anatomical opening O has been identified and/or at least partially opened by the fluid 120 delivered by the fluid delivery device 1100, such as to further enlarge the opening O.

Various procedures which may be performed with a device and/or system formed in accordance with various principles of the present disclosure may use a contrast agent. The fluid delivery device 1100 accordingly may include a contrast-agent-delivery lumen 1128 configured to be in fluid communication with a source of a contrast agent and to deliver the contrast agent to the target site TS and/or to the anatomical structure beyond the opening O opened with the use of the fluid delivery device 1100. The contrast agent may be supplied to the contrast-agent-delivery lumen 1128 via a syringe attached to an appropriate port or connector (e.g., a Luer connector) on the handle 1140 of the fluid delivery device 1100, and/or the handle 1240 of the delivery device 1200 through one or more other suitable connections. The contrast agent may be delivered to the target site TS and may be used to temporarily improve imaging within the patient by, for example, x-ray, fluoroscopy, computed tomography (CT), or magnetic resonance (MR) imaging, ultrasound, and the like.

It will be appreciated that accessing a body lumen substantially without contact by a medical device and allowing insertion of the guidewire directly into the lumen substantially without or without contacting the body lumen opening, such as described above, may decrease both tissue abrasion and lumen compression when compared to known medical devices and techniques. In various embodiments, devices, systems and methods of the present disclosure allow atraumatic contact and/or entry of a guidewire and/or an endoscopic accessory tool with or into generally narrow anatomical structures through controlled reduction or elimination of friction by using fluid at the distal end of the access device to move or open the lumen and/or surrounding tissue substantially without or without device contact.

All apparatuses and methods discussed herein are examples of apparatuses and/or methods implemented in accordance with one or more principles of this disclosure. It is to be understood by one of ordinary skill in the art that the present discussion is a description of illustrative examples of embodiments only, and is not intended as limiting the broader aspects of the present disclosure. The examples described herein are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. Moreover, various further benefits of the various aspects, features, components, and structures of devices, systems, and methods such as described above, in addition to those discussed above, may be appreciated by those of ordinary skill in the art.

Although embodiments of the present disclosure may be described with specific reference to medical devices and systems and procedures for treating the gastrointestinal system, it should be appreciated that such medical devices, systems, and methods may be used to treat tissues of the abdominal cavity, digestive system, urinary tract, reproductive tract, respiratory system, cardiovascular system, circulatory system, and the like, and in various ductal, luminal, vascular, or other body lumen anatomies. Any of a variety of medical devices, instruments, tools, etc., may be used in conjunction with the devices and systems of the present disclosure, including, without limitation, endoscopes, duodenoscopes, catheters, ureteroscopes, bronchoscopes, colonoscopes, arthroscopes, cystoscopes, hysteroscopes, cholangioscopes, etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A fluid delivery device configured to deliver pressurized fluid through a fluid delivery port at an end thereof to a target site within a patient, wherein:
said fluid delivery device defines therethrough a fluid delivery lumen fluidly couplable with a pressurized fluid source, and at least a first additional lumen configured for delivery of one of a device, a contrast fluid, a guidewire, or a wire filament configured for cutting tissue; and
a side orifice through a wall of said fluid delivery device defining said fluid delivery lumen, the side orifice defined proximal to the fluid delivery port and adjacent thereto to provide pressure relief upon partial or complete obstruction of the fluid delivery port.

2. The fluid delivery device of claim 1, wherein the fluid delivery lumen extends longitudinally along said fluid delivery device and said fluid delivery port extends out a distal end of said fluid delivery device to deliver fluid longitudinally out the fluid delivery device.

3. The fluid delivery device of claim 1, wherein the fluid delivery lumen has a first diameter from a proximal end of the fluid delivery device to a narrowed region within the fluid delivery lumen having a second diameter smaller than the first diameter.

4. The fluid delivery device of claim 3, wherein the narrowed region is upstream of the side orifice.

5. The fluid delivery device of claim 4, wherein the narrowed region creates a Venturi effect causing entrainment of fluid at the target site into the side orifice.

6. The fluid delivery device of claim 5, wherein the entrained fluid is redirected back out the fluid delivery port of the fluid delivery system.

7. The fluid delivery device of claim 1, wherein the first additional lumen is configured for passage of a guidewire therethrough and a further instrument over the guidewire to the target site.

8. The fluid delivery device of claim 1, wherein a second additional lumen is defined through said fluid delivery device configured for delivery of one of a device, a contrast fluid, a guidewire, or a wire filament configured for cutting tissue.

9. The fluid delivery device of claim 8, wherein a third additional lumen is defined through said fluid delivery device configured for delivery of one of a device, a contrast fluid, a guidewire, or a wire filament configured for cutting tissue.

10. The fluid delivery device of claim 9, wherein:
the first additional lumen is configured for passage of a guidewire therethrough and a further instrument over the guidewire;
the second additional lumen is configured for passage therethrough of a wire filament configure for cutting tissue; and
the third additional lumen is configured for delivery therethrough of a contrast fluid to the target site.

11. The fluid delivery device of claim 1, wherein the side orifice is in fluid communication with a return lumen to provide pressure relief upon partial or complete obstruction of the fluid delivery port by delivering fluid outside the patient via the return lumen.

12. A system for performing a procedure past an orifice within a patient's body, said system comprising:
a fluid delivery device defining a fluid delivery lumen therethrough ending at a distal fluid delivery port, the fluid delivery device having a side port proximal to the fluid delivery port and adjacent thereto to provide pressure relief upon partial or complete obstruction of the fluid delivery port; and
an additional instrument configured to be inserted along the fluid delivery device and inserted through the orifice within the patient's body.

13. The system of claim 12, further comprising a delivery device comprising a tubular delivery element configured to deliver said fluid delivery device therethrough.

14. The system of claim 12, wherein:
the fluid delivery lumen is couplable with a pressurized fluid source; and
the diameter of the fluid delivery lumen decreases upstream and adjacent the side port to create a Venturi effect causing entrainment of fluid at the target site into the side port.

15. The system of claim 12, wherein the fluid delivery device defines an additional lumen therethrough for delivery of the additional instrument therethrough.

16. A method of regulating fluid delivered to a target site within a patient, said method comprising:
advancing a distal end of a fluid delivery device to the target site;
directing a fluid delivery port toward tissue at the target site;
delivering fluid through the fluid delivery port; and
regulating the fluid delivered through the fluid delivery port via a side port defined in the fluid delivery device.

17. The method of claim 16, wherein regulating the fluid delivered through the fluid delivery port comprises diverting fluid through the side port upon the fluid deliver port contacting tissue at the target site to relieve fluid pressure on the contacted tissue.

18. The method of claim 16, wherein the diameter of the fluid delivery lumen is tapered upstream of the side port and regulating the fluid delivered through the fluid delivery port comprises entraining fluid delivered by the fluid delivery port to the target site into the side port and recycling the entrained fluid out the fluid delivery port.

19. The method of claim 16, further comprising delivering fluid through the fluid delivery port to cause an opening in a tissue wall at the target site to dilate.

20. The method of claim 19, further comprising delivering an instrument to the dilated opening in the tissue wall and through the dilated opening.
